(19) United States
(12) Patent Application Publication
(10) Pub. No.: US 2008/0015522 A1
(43) Pub. Date: Jan. 17, 2008

(54) Dual Chamber Injector Integrated With Micro-Needles

(75) Inventors: Yehoshua Yeshurun, Haifa (IL); Yotam Levin, Nes Ziona (IL); Gilad Lavi, Rishon Lezion (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: NANOPASS TECHNOLOGIES LTD., Nes Ziona (IL)

(21) Appl. No.: 11/775,875

(22) Filed: Jul. 11, 2007

Related U.S. Application Data
(60) Provisional application No. 60/806,934, filed on Jul. 11, 2006.

(51) Int. Cl. A61M 5/32

(52) U.S. Cl. 604/272

(57) ABSTRACT

This invention relates to a medical injection system for the intradermal injection of two or more fluid substances via micro-needles, and most preferably via a linear array of micro-needles, although the array need not be linear. Injecting two fluid substances contained in two separate chambers of the delivery device can be done by various methods. These methods may be generally categorized as, a) sequential injection, b) simultaneous injection, c) parallel injection and d) closed-loop injection processes. The present invention includes devices configured to perform all three of these methods.

### FIELD AND BACKGROUND OF THE INVENTION

The present invention relates to medical injection systems and, in particular, it concerns a medical injection system for the intradermal injection of two or more fluid substances via micro-needles.

Many medical formulations include more than one fluid substance, and in some cases, mixing all components together results in limited stability, limited shelf life or otherwise deterioration of efficacy and/or activity of the substances in the resulting mixture. This obstacle is quite common in mixing formulations which include an active ingredient (such as a vaccine) which should be mixed with a chemical adjuvant, which may or may not have different biochemical and/or physical features such as, but not limited to, lipophylic/hydrophilic index, pH, and osmolarity.

A number of syringe devices configured for mixing fluid substances just prior to delivery have been suggested. Typically, these devices include two chambers configured such that the contents of one chamber are emptied into the other chamber for mixing, as represented by U.S. Pat. No. 6,417,656 to Vetter et al., WO2005/039669 and WO00/53244. All of these devices suffer from necessarily complicated mechanisms for sealed transference of at least one fluid from one chamber to another for mixing.

Further, none of these syringe devices is configured for intradermal injection via micro-needles.

There is therefore a need for a medical injection system for the intradermal injection of two or more fluid substances via micro-needles.

### SUMMARY OF THE INVENTION

The present invention is a medical injection system for the intradermal injection of two or more fluid substances via micro-needles.

According to the teachings of the present invention there is provided, an injection device for introduction of fluid into biological tissue, the injection device comprising: a) at least first and second chambers configured for the storage of at least first and second fluid substances respectively; b) at least one plunger element operative to expel the first and second fluid substances from the first and second chambers; c) a micro-needle injection interface attached to a delivery end of the injection device; and e) at least one fluid flow passageway providing fluid communication between the first and second chambers and the micro-needle injection interface.

According to a further teaching of the present invention, the micro-needle injection interface is a micro-needle chip constructed using MEMS technology.

According to a further teaching of the present invention, the micro-needle injection interface includes at least one micro-needle formed from at least one material chosen from a list including, silicon and silicon dioxide, metal and metal alloys, polymers, glass and combinations thereof.

According to a further teaching of the present invention, the micro-needle injection interface includes at least one hollow micro-needle.

According to a further teaching of the present invention, the first and second chambers are disposed in series such that operation of a single plunger element is effective to expel the first and second fluid substances sequentially from the first and second chambers.

According to a further teaching of the present invention, the first and second chambers are configured such that expulsion of the first fluid substance from the first chamber is achieved by operation of a first plunger element and expulsion of the second fluid substance from the second chamber is achieved by operation of a second plunger element, the at least one fluid flow passageway being configured as two fluid flow passageways such that one fluid passageway extends from each of the first and second chambers.

According to a further teaching of the present invention, the two fluid flow passageways join in at least one region in which the first and second fluid substances are mixed and the resulting mixture is injected through all micro-needles in the micro-needles injection interface.

According to a further teaching of the present invention, the two fluid flow passageways are configured such that each fluid flow passageway is in fluid communication with a number of dedicated micro-needles configured in the micro-needles injection interface.

According to a further teaching of the present invention, the fluid flow passages are formed as part of a block of polymer material, and wherein the micro-needles injection interface includes a substrate supporting a plurality of micro-needles, the polymer block and the substrate being formed with interlocking features to facilitate sealed connection of the substrate to the polymer material.

According to a further teaching of the present invention, the interlocking features are configured to facilitate formation of a seal line subdividing the plurality of micro-needles into two subgroups, each of the first and second flow passageways being in fluid interconnection with a corresponding subgroup of the micro-needles.

There is also provided according to the teachings of the present invention, a method for introduction of more than one fluid substance into biological tissue, the method comprising: a) providing an injection device configured such that a single injection operation expels at least first and second fluid substances stored separately within the injection device, the injection device having a micro-needles injection interface through which the first and second fluid substances are expelled; b) penetrating the biological tissue with micro-needles extending from the micro-needles injection interface; and c) operating the injection device so as to expel the first and second fluid substances so as to inject the at least first and second fluid substances into the biological tissue.

According to a further teaching of the present invention, the injection device is implemented so as to include: a) at least first and second chambers configured for the storage of the first and second fluid substances respectively; b) at least one plunger element operative to expel the first and second fluid substances from the first and second chambers; and c) at least one fluid flow passageway providing fluid communication between the first and second chambers and the micro-needle injection interface.

According to a further teaching of the present invention, there is also provided constructing the micro-needle injection interface as a micro-needle chip using MEMS technology.

According to a further teaching of the present invention, the micro-needle injection interface is implemented with at least one hollow micro-needle.

According to a further teaching of the present invention, the operating of the injection device is such that the first and second fluid substances are injected sequentially.

According to a further teaching of the present invention, the operating of the injection device is such that the first and second fluid substances are injected simultaneously.

According to a further teaching of the present invention, the simultaneous injection is such that the first and second fluid substances coalesce within the injection device prior to injection.

According to a further teaching of the present invention, the operating of the injection device is such that the first and second fluid substances are injected in parallel.

According to a further teaching of the present invention, the parallel injection is such that the first and second fluid substances coalesce within the biological tissue subsequent to injection.

According to a further teaching of the present invention, the parallel injection is such that the first and second fluid substances are injected into the skin in two discrete locations and do not readily coalesce.

According to a further teaching of the present invention, there is also provided forming the fluid flow passages as part of a block of polymer material, and wherein the micro-needles injection interface includes a substrate supporting a plurality of micro-needles, the polymer block and the substrate being formed with interlocking features to facilitate sealed connection of the substrate to the polymer material.

According to a further teaching of the present invention, the operating of the injection device is such that the first and second fluid substances are injected intradermally.

According to a further teaching of the present invention, the operating of the injection device is implemented so as to perform a closed-loop injection process

### BRIEF DESCRIPTION OF THE DRAWINGS

The invention is herein described, by way of example only, with reference to the accompanying drawings, wherein:

FIG. 1 is a transparent isometric view of a first preferred embodiment of a syringe device equipped with micro-needles adapter, constructed and operational according to the teachings of the present invention, enabling the sequential injection of two fluid substances;

FIG. 1A is a detail of FIG. 1;

FIG. 2A, FIG. 2B and FIG. 2C are isometric cross-section views of the embodiment of FIG. 1 in different functional positions, wherein FIG. 2A shows the device before use, FIG. 2B shows the device after the first component was delivered, FIG. 2C shows the device when delivery is completed;

FIG. 3 is a detail of FIG. 2B;

FIG. 4 is an isometric view of a second preferred embodiment of a syringe device equipped with micro-needles adapter, constructed and operational according to the teachings of the present invention, enabling the parallel injection of two fluid substances;

FIG. 5 is an isometric view of the embodiment of FIG. 4 before the introduction of pre-filled syringes;

FIG. 6 is a local cross-section view of the embodiment of FIG. 4;

FIG. 6A is a detail of FIG. 6;

FIG. 7A is a transparent bottom isometric view of a third preferred embodiment of a micro-needles adapter, constructed and operational according to the teachings of the present invention, enabling the parallel injection of two fluid substances;

FIG. 7B is an opaque bottom isometric view of the embodiment of FIG. 7A; and

FIG. 8 is a schematic illustration of an attachment structure for the attachment of a micro-needles chip to a syringe device.

### DESCRIPTION OF THE PREFERRED EMBODIMENTS

The present invention is a medical injection system for the intradermal injection of two or more fluid substances via micro-needles.

The principles and operation of a medical injection system according to the present invention may be better understood with reference to the drawings and the accompanying description.

By way of introduction, the medical injection system of the present invention includes a micro-needles injection interface, typically manufactured by MEMS technology as a micro-needles chip. The micro-needles may be formed from any suitable material including, but not limited to, silicon and silicon dioxide, metal and metal alloys, polymers, glass and combinations thereof. Details of preferred implementations of micro-needle structures and associated delivery devices not explicitly addressed in this document may be found in the following patent publications which are hereby incorporated by reference in their entirety as if fully set out herein: U.S. Pat. No. 6,533,949, US 2005/0165358, WO 2005/049107 and US 2005/0209566.

This invention relates to a medical injection system for the intradermal injection of two or more fluid substances via micro-needles, and most preferably via a linear array of micro-needles, although the array need not be linear.

Injecting two fluid substances contained in two separate chambers of the delivery device can be done by various methods. These methods may be generally categorized as, a) sequential injection, b) simultaneous injection, c) parallel injection, and d) close-loop injection processes. The present invention includes devices configured to perform all three of these methods.

As used herein, the term “sequential injection” refers to the injection of a first substance followed by the injection of a second substance. The sequential injection may be by way of a common delivery path through a common micro-needle or array of micro needles such that the substances are delivered to the same location. Alternatively, the sequential injection may be by way of dual delivery paths through separate micro-needles or arrays of micro-needles such that the substances are delivered to the different locations in close proximity.

As used herein, the term “simultaneous injection” refers to the injection of a mixture of substances that are mixed in the delivery device just prior to injection, and are thereby delivered simultaneously through a common micro-needle or array of micro needles to the same location.

As used herein, the term “parallel injection” refers to the substantially simultaneous injection of two different substances by way dual delivery paths through separate micro-needles or arrays of micro-needles such that the substances are delivered to the tissue at different locations in close proximity. The proximity and physical and/or chemical properties of the substances injected in parallel may or may not result in the substances mixing within the biological tissues into which they are injected.

As used herein, the term “closed-loop injection process” refers to processes in which fluid is withdrawn from the tissue through one or more micro-needles and fluid and/or a therapeutic substance is injected through one or more other micro-needles. Such process is of particular benefit in the delivery of insulin. In a closed-loop process using the injection device of the present invention, body fluid may be drawn from the tissue into the device via a first set of micro-needles, the glucose level measured and the proper dose of insulin delivered via a second set of micro needles without removing the injection device from the patient.

As mentioned above, many medical formulations include more than one fluid substance, and in some cases, mixing all components together results in limited stability, limited shelf life or otherwise deterioration of efficacy and/or activity of the mixed substance. This obstacle is quite common in mixing formulations including an active ingredient (such as a vaccine) which should be mixed with adjuvants, which may or may not have different biochemical and/or physical features such as, but not limited to, lipophylic/hydrophilic index, pH, and osmolarity.

Other applications that will benefit from the present invention include immunomodulation, and dermal filling. The devices and methods discussed herein are well suited for use with hollow micro-needles. That is to say, micro-needles that are configured with a fluid flow bore through at least a portion of their height.

The above mentioned methods are well suited for use with a wide range of substances as exemplified in the following list.
1. Active substances in a first chamber and various formulation agents in a second chamber. By non-limiting example, materials that are not stabile together or are difficult to formulate together.
2. Vaccines in a first chamber and adjuvants in a second chamber.
3. A first vaccine(s) in a first chamber and additional vaccine(s) in a second chamber.
4. A medication in a first chamber and an anesthetic that can reduce the pain of delivery in a second chamber. By non-limiting example, a dermal filler and lidocaine, where lidocaine is delivered first, numbs the area to reduce pain related to the pressure of injecting dermal fillers. Another non-limiting example is the delivery of adrenalin and a therapeutic substance locally, where adrenalin constricts blood vessels and slows down systemic distribution of thattherapeutic substance.
5. Two or more substances that are unstable together but are synergistic in general. By non-limiting example, lidocaine and bicarbonate can be mixed immediately prior to injection. The bicarbonate reduces the acidity of the lidocaine formulation and thus reduces the burning sensation. However, the two cannot be mixed and then left together for long periods of time.
6. Delivery of a substance with a bulking agent that can reduce delivery rates. By nonlimiting example, an active substance together with a biodegradable polymer such as, but not limited to, PEG that reduces or slows down degradation of the active substance. It will be appreciated that this mixture may be delivered in a jell-like consistency. Practical testing of the devices of the present invention has shown positive results relating to the injection of jells.

The use of the injection device of the present invention provides numerous advantages over the devices of the prior art. Among the advantages are the reduction of time required to inject two or more substances; a lessening of apprehension on the part of the patient, especially children; a reduction in pain to the patient due to fewer injections; a reduction in the chance of local trauma and misuse of the subject tissue; and a reduction in the chance of needle pricks occurring to the medical staff due to fewer injections being made.

A further advantage is that there is no need to test the long term stability of a mixture of components since they are shelved separately in two discrete chambers. This also may reduce the overall development time and expenses for new or old drugs.

Still a further advantage of the injection system of the present invention is that it is beneficial for use when the reaction that results from the mixture of the substances is preferably done in the body. This is similar to the concept of bio-glue which is applied locally on the skin, and intended to have a reaction in the location of treatment only.

The injection system of the present invention is also advantageous for use with substances that aggregate or for some other reason one substance impedes the activity or the stability of the other substance, such as, by non limiting example, lidocaine and bicarbonate.

The injection system of the present invention provides an injection device that enables the injection of two component substances stored in two independent chambers.

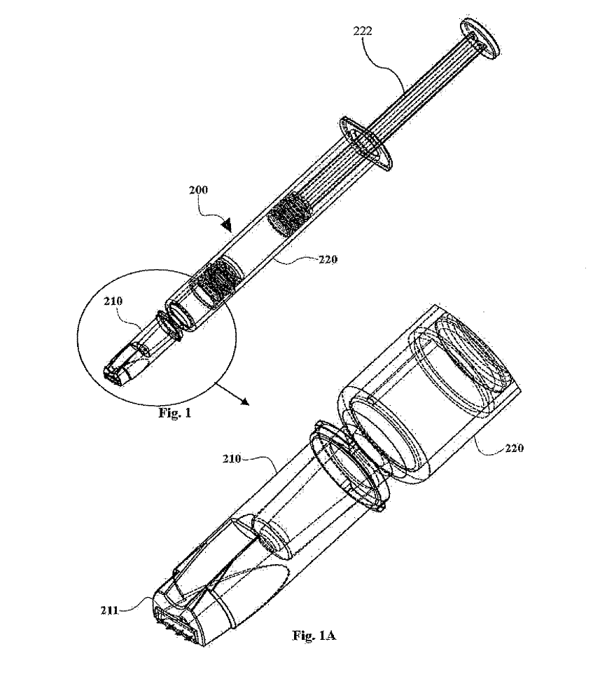

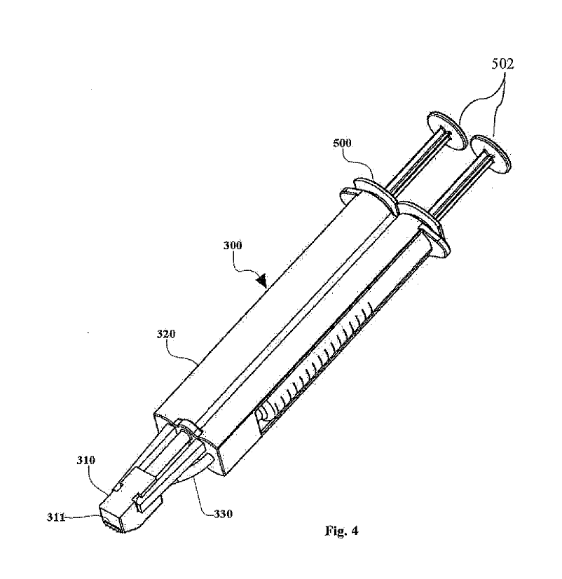

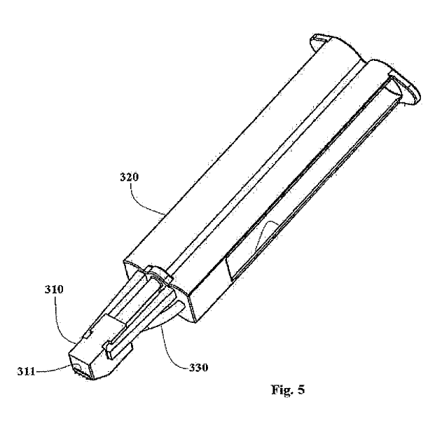

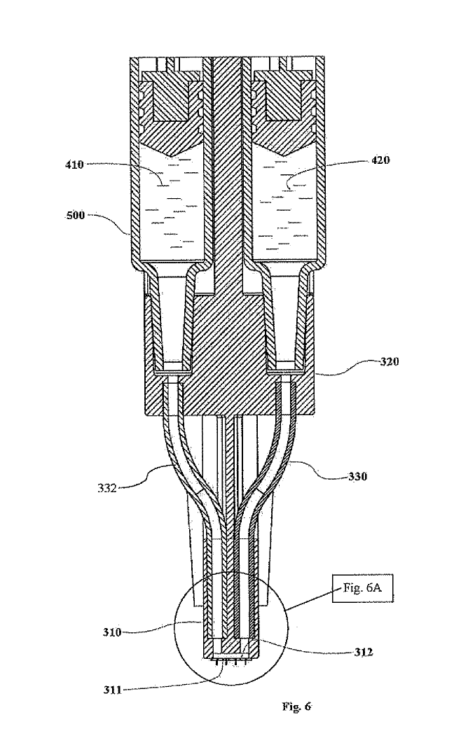

Discussed herein, are a number of different non-limiting embodiments of the injection device. An injection device having dual chambers configured in series such that delivery of the substances is responsive to operation of a single plunger element and the substances are delivered sequentially is discussed with regard to FIGS. 1-3. An injection device having dual chambers configured not in series, and preferably in parallel, such that the fluid substance stored in each chamber is expelled by the operation of a separate plunger element is discussed with regard to FIGS. 4-7B. Using variants of this embodiment, substances may be delivered sequentially, simultaneously, or in parallel. In all cases, the fluid substances are delivered by a single injection operation of the injection device of the present invention.

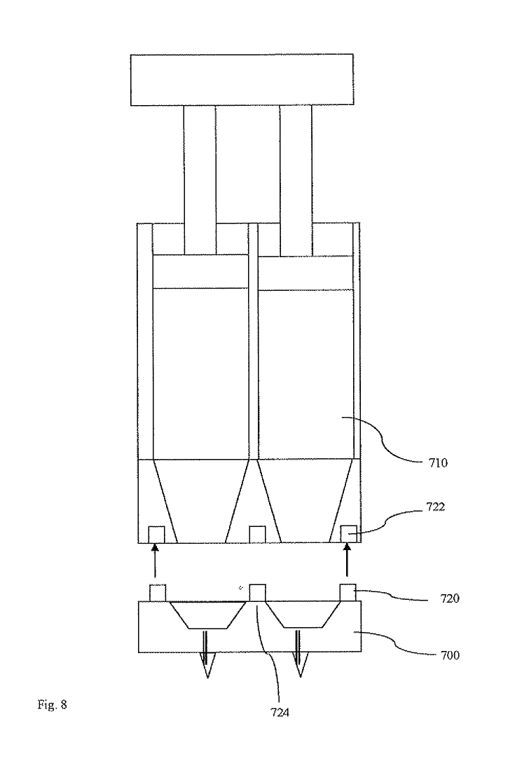

An attachment configuration well suited for the attachment of the micro-needle chip to the tip element of the injection device of the present invention is discussed with regard to FIG. 8.

Referring now to the drawings, FIGS. 1-3 illustrate a first preferred embodiment of an injection device of the present invention, generally referred to as 200, having dual chambers configured in series such that delivery of the fluid substances stored therein is responsive to operation of a single plunger element and the fluid substances are delivered sequentially by a single stroke of the plunger. That is to say, the fluid substances are delivered by a single injection operation of the injection device. As illustrated here, injection device 200 includes a dual chamber syringe body 220, a plunger element 222 and a micro-needles adapter 210 attached to a delivery end of the syringe body 220. The micro-needles adapter 210 includes a micro-needles chip 211 configured with an array of micro-needles.

Syringe 220 is configured with two chambers, a proximal chamber 230 and a distal chamber 240. The proximal chamber is filled with fluid substance 250 while the distal chamber is filled with fluid substance 260. The injection device 200 also includes two pistons. Piston 231 is attached to the plunger element 222 and applies pressure to the proximal chamber 230 when actuated. Piston 241, which also acts as the separation wall between the proximal chamber 230 and distal chamber 240, is free floating and actuated by fluid pressure applied to it by fluid substance 250 when plunger element 222 actuates piston 231.

Operational delivery fluid substances 250 and 260 by a single stroke of the plunger element 222 is as follows. Micro-needles adapter 210 is attached to the injection site and the micro-needles configured in the micro-needles chip 211 penetrate the skin of the patient. Plunger element 222 is pushed into the syringe body 220, thereby moving piston 231 toward the distal or delivery end of the syringe body 220. The movement of piston 231 applies pressure to fluid substance 250 which in turn generates distal movement of piston 241 that in turn expels fluid substance 260 through the micro-needles adapter 210 and micro-needles configured in the micro-needles chip 211 thereby delivering it to the injection site.

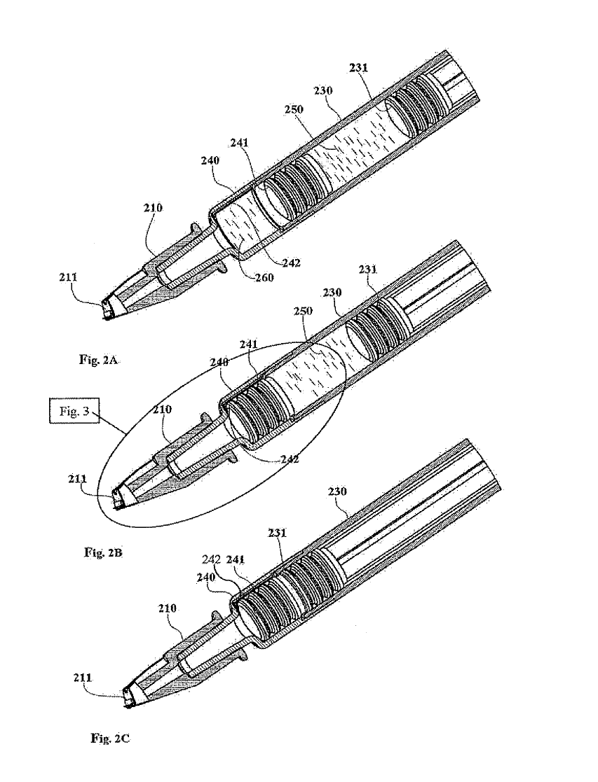

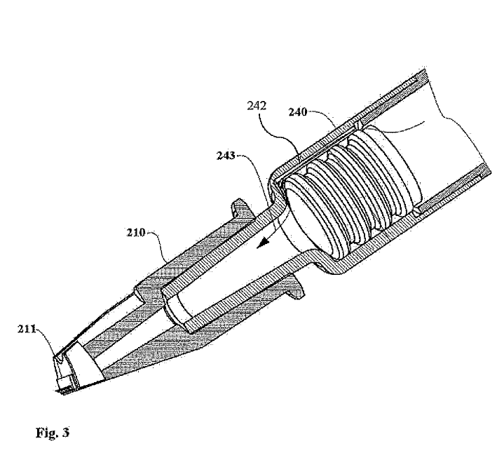

At least one by-pass groove 242 is configured on the inner surface of the distal end of the wall of the syringe body 220. This portion of the syringe body 220 is initially associated with chamber 240. Once piston 240 reaches the distal wall of the syringe body 220 as illustrated in FIGS. 2B and 3, the pressure applied to fluid substance 250 by plunger element 222 forces fluid substance 250 through by-pass groove 242, the micro-needles adapter 210 and micro-needles configured in the micro-needles chip 211 thereby delivering it to the injection site. Arrow 243 in FIGS. 2B and 3 illustrates the path of fluid substance 250 through by-pass groove 241.

FIGS. 4-6 illustrate a second preferred embodiment of an injection device of the present invention, generally referred to as 300. Injection device 300 includes two major components, the syringe housing 320, into which pre-filled syringes 500 are inserted, and the micro-needles adapter 310 that includes micro-needles chip 311 configured with an array of micro-needles. Syringes 500 are connected to housing 320 via 2 female luer connectors.

It will be appreciated that the engagement of syringes 500 with syringe housing 320 is done manually and that any method of titration or air purge is manually performed by the operator prior to insertion of the syringe 500 into the syringe housing 320.

During the injection process the operator will apply pressure to each of the plunger elements 502 extending from each of the syringes 500, preferably with the thumb or other finger used for pushing. The typical distance between the centers of both plungers 502 is at the magnitude of 11 mm, which is reasonable to be covered by an adult's thumb. That pressure applied will prevent any attempt of one fluid substance to flow into the other chamber rather than exit the device via the micro-needles. In this manner, the fluid substances in both of the syringes are delivered by a single injection operation of the injection device of the present invention.

In this embodiment, fluid communication between syringe housing 320 and the micro-needles adapter 310 is provided via fluid passageways in flexible tubing 330 and 332. During operation, fluid substances 410 and 420, which are stored in their respective syringes 500, are expelled from syringes 500 by the movement of plungers 502. Fluid substances 410 and 420 each flow through respective independent fluid passageways provided by flexible tubing 330 and 332 until they reach region 312 in the micro-needles adapter 310. Region 312 provides access to the full array of micro-needles in the micro-needles chip 311 to the fluid substances in region 312. In the embodiment illustrated here, region 312 functions as a manifold in which the two fluid substances 410 and 420 are mixed just prior to injection via the micro-needles configured in the micro-needles chip 311.

It will be readily understood that this configuration of micro-needles adapter 310 provides simultaneous injection of the two fluid substances 410 and 420.

An alternative configuration of the micro-needles adapter 310 that provides parallel injection of fluid substances 410 and 420 may include a barrier in region 312 that limits the number of micro-needles accessible to each of the fluid substances. Alternatively two separate micro-needle chips may be used, with each micro-needle chip being dedicated to each of the separate fluid passageways provided by flexible tubing 330 and 332. Substances injected using this configuration of micro-needles adapter 310 will be mixed only after penetrating the injection site. It should be noted that sequential injection of fluid substances 410 and 420 may also be achieved using this alternative configuration of micro-needles adapter 310 by sequential operation of plungers 502. Here too, the fluid substances are delivered by a single injection operation of the injection device of the present invention.

For embodiments having two or more plunger elements, it will be appreciated that an optional mechanical link element may be used to interconnect the plungers. Such a mechanical link element would be of particular benefit during parallel injections.

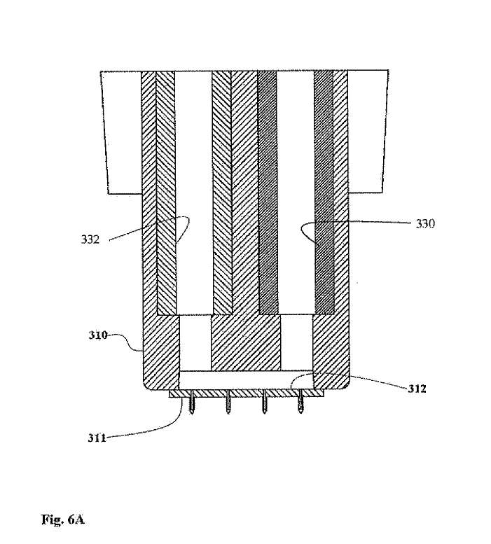

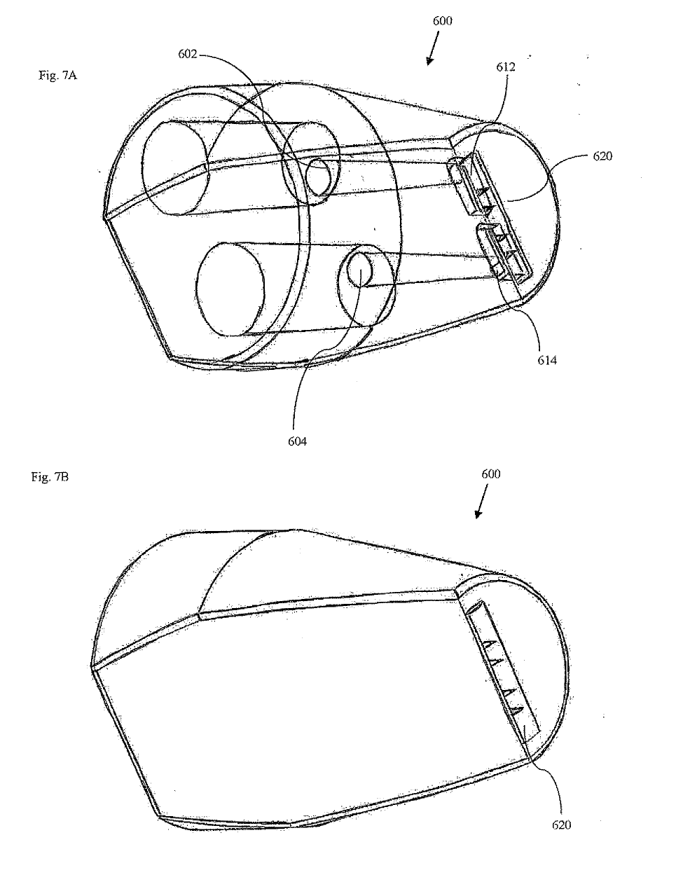

FIGS. 7A and 7B illustrate a third preferred embodiment of a micro-needles adapter 600 for use with a syringe housing similar to syringe housing 310 in embodiment 300. As illustrated here, each of the fluid passageways 602 and 604 terminate in separate manifold regions 612 and 614 respectively. These manifold regions 612 and 614 provide fluid access to a limited number of micro-needles configured in the micro-needle chip 620. That is to say, each of the fluid flow passageways are configured such that each fluid flow passageway 602 and 604 is in fluid communication with a number of dedicated micro-needles configured in said micro-needles chip. This configuration of the micro-needles adapter 600 provides parallel injection of fluid substances.

It will be readily appreciated that an alternative configuration of micro-needles adapter 600 that provides simultaneous injection of fluid substance is possible by providing a single manifold region fed by both fluid passageways 602 and 604 and providing fluid access to the full array of micro-needles configured in the micro-needle chip 620.

FIG. 8 schematically illustrates a preferred attachment configuration for facilitating the attachment of a micro-needles chip 700 to the tip of a syringe housing 710.

Generally speaking, the fluid flow passages are formed as part of a block of polymer material and the micro-needles injection interface includes a substrate supporting a plurality of micro-needles. The polymer block and the substrate are, therefore, formed with interlocking features to facilitate sealed connection of the substrate to the polymer material. In some applications, the interlocking features are configured to facilitate formation of a seal line subdividing the plurality of micro-needles into two subgroups, each of the first and second flow passageways being in fluid interconnection with a corresponding subgroup of the micro-needles. A non-limiting example of which is discussed herebelow

As illustrated here, micro-needles chip 700 is configured with a plurality of attachment ridges 720 and syringe housing 710 is configured with corresponding grooves 722 into which ridges 720 are inserted when micro-needles chip 700 is attached to syringe housing 710. This attachment configuration assists with alignment and stability of attachment of the micro-needles chip 700 as it is attached to syringe housing 710, as well as facilitating formation of a seal line in region 724. It will be appreciated that the attachment ridges may be configured in the syringe housing and the corresponding grooves may be configured in the micro-needles chip. In the case of MEMS manufacturing techniques, the back-side of the micro-needles chip may be processed by wet etching techniques to form the grooves. The complementary ridges in the surface of the polymer block for engaging the grooves can be formed by micro injection-molding techniques. It should be noted that this attachment configuration may also be used to attach a micro-needles chip to a micro-needles adapter, such as those illustrated in FIGS. 1-7B.

It will be appreciated that the above descriptions are intended only to serve as examples and that many other embodiments are possible within the spirit and the scope of the present invention.

What is claimed is:

1. An injection device for introduction of fluid into biological tissue, the injection device comprising:
(a) at least first and second chambers configured for the storage of at least first and second fluid substances respectively,
(b) at least one plunger element operative to expel said first and second fluid substances from said first and second chambers;
(c) a micro-needle injection interface attached to a delivery end of the injection device; and
(d) at least one fluid flow passageway providing fluid communication between said first and second chambers and said micro-needle injection interface.

2. The injection device of claim 1, wherein said micro-needle injection interface is a micro-needle chip constructed using MEMS technology.

3. The injection device of claim 1, wherein said micro-needle injection interface includes at least one micro-needle formed from at least one material chosen from a list including, silicon and silicon dioxide, metal and metal alloys, polymers, glass and combinations thereof.

4. The injection device of claim 1, wherein said micro-needle injection interface includes at least one hollow micro-needle.

5. The injection device of claim 1, wherein said first and second chambers are disposed in series such that operation of a single plunger element is effective to expel said first and second fluid substances sequentially from said first and second chambers.

6. The injection device of claim 1, wherein said first and second chambers are configured such that expulsion of said first fluid substance from said first chamber is achieved by operation of a first plunger element and expulsion of said second fluid substance from said second chamber is achieved by operation of a second plunger element, said at least one fluid flow passageway being configured as two fluid flow passageways such that one fluid passageway extends from each of said first and second chambers.

7. The injection device of claim 6, wherein said two fluid flow passageways join in at least one region in which said first and second fluid substances are mixed and the resulting mixture is injected through all micro-needles in said micro-needles injection interface.

8. The injection device of claim 6, wherein said two fluid flow passageways are configured such that each fluid flow passageway is in fluid communication with a number of dedicated micro-needles configured in said micro-needles injection interface.

9. The injection device of claim 6, wherein said fluid flow passages are formed as part of a block of polymer material, and wherein said micro-needles injection interface includes a substrate supporting a plurality of micro-needles, said polymer block and said substrate being formed with interlocking features to facilitate sealed connection of said substrate to said polymer material.

10. The injection device of claim 9, wherein said interlocking features are configured to facilitate formation of a seal line subdividing said plurality of micro-needles into two subgroups, each of said first and second flow passageways being in fluid interconnection with a corresponding subgroup of said micro-needles.

11. A method for introduction of more than one fluid substance into biological tissue, the method comprising:
(a) providing an injection device configured such that a single injection operation expels at least first and second fluid substances stored separately within said injection device, said injection device having a micro-needles injection interface through which said first and second fluid substances are expelled;
(b) penetrating the biological tissue with micro-needles extending from said micro-needles injection interface; and
(c) operating said injection device so as to expel said first and second fluid substances so as to inject said at least first and second fluid substances into the biological tissue.

12. The method of claim 11, wherein said injection device is implemented so as to include:
(a) at least first and second chambers configured for the storage of said first and second fluid substances respectively;
(b) at least one plunger element operative to expel said first and second fluid substances from said first and second chambers; and
(c) at least one fluid flow passageway providing fluid communication between said first and second chambers and said micro-needle injection interface.

13. The method of claim 11, further including constructing said micro-needle injection interface as a micro-needle chip using MEMS technology.

14. The method of claim 11, wherein said micro-needle injection interface is implemented with at least one hollow micro-needle.

15. The method of claim 11, wherein said operating of said injection device is such that said first and second fluid substances are injected sequentially.

16. The method of claim 11, wherein said operating of said injection device is such that said first and second fluid substances are injected simultaneously.

17. The method of claim 16, wherein said simultaneous injection is such that said first and second fluid substances coalesce within the injection device prior to injection.

18. The method of claim 11, wherein said operating of said injection device is such that said first and second fluid substances are injected in parallel.

19. The method of claim 18, wherein said parallel injection is such that said first and second fluid substances coalesce within the biological tissue subsequent to injection.

20. The method of claim 18, wherein said parallel injection is such that said first and second fluid substances are injected into the skin in two discrete locations and do not readily coalesce.

21. The method of claim 11, further including forming said fluid flow passages as part of a block of polymer material, and wherein said micro-needles injection interface includes a substrate supporting a plurality of micro-needles, said polymer block and said substrate being formed with interlocking features to facilitate sealed connection of said substrate to said polymer material.

22. The method of claim 11, wherein said operating of said injection device is such that said first and second fluid substances are injected intradermally.

23. The method of claim 11, wherein one of said first and second fluid substances is implemented as a vaccine.

24. The method of claim 11, wherein said operating of said injection device is implemented so as to perform a closed-loop injection process.
